(19) United States
(12) Patent Application Publication
(10) Pub. No.: US 2024/0366597 A1
(43) Pub. Date: Nov. 7, 2024

(54) CANCER TREATMENT COMBINATIONS

(71) Applicant: Viracta Subsidiary, Inc., Cardiff, CA (US)

(72) Inventors: Lisa ROJKJAER, Cardiff, CA (US); Robert MCRAE, Solana Beach, CA (US)

(21) Appl. No.: 18/580,500

(22) PCT Filed: Jul. 20, 2022
(86) PCT No.: PCT/US2022/037755

§ 371 Date: Jan. 18, 2024

Related U.S. Application Data
(60) Provisional application No. 63/224,346, filed on Jul. 21, 2021.

(51) Int. Cl. A61K 31/506; A61K 31/522; C07K 16/28

(52) U.S. Cl. CPC A61K 31/506; A61K 31/522; C07K 16/2818; C07K 16/2827

(57) ABSTRACT

Described herein is a method of treating a cancer or tumor in an individual, the method comprising administering to the individual afflicted with the cancer or tumor an effective amount of: a) nanatinostat; b) a nucleoside analog; c) and a checkpoint inhibitor antagonist.

### CROSS REFERENCE TO RELATED APPLICATIONS

This Application claims the benefit of U.S. Provisional App. Ser. No. 63/224,346 filed on Jul. 21, 2021, which is incorporated by reference herein in its entirety.

### SUMMARY

In one aspect described herein is a method of treating a cancer or tumor in an individual, the method comprising administering to the individual afflicted with the cancer or tumor an effective amount of: a) a histone deacetylase inhibitor (HDACi); b) a nucleoside analog; c) and a checkpoint inhibitor antagonist. In certain embodiments, the HDACi comprises vorinostat, romidepsin, mocetinostat, belinostat, pracinostat, givinostat, panobinostat, CUDC-101, zabinostat, chidamide, domatinostat, entinostat, and combinations thereof. In certain embodiments, the HDACi comprises nanatinostat. In certain embodiments, the nanatinostat is administered at a dose of about 5 milligrams to about 160 milligrams per day. In certain embodiments, the nanatinostat is administered at a dose of about 5 milligrams to about 80 milligrams per day. In certain embodiments, the nanatinostat is administered at a dose of about 5 milligrams to about 40 milligrams per day. In certain embodiments, the nanatinostat is administered at a dose of about 5 milligrams to about 20 milligrams per day. In certain embodiments, the nanatinostat is administered at a dose of about 10 milligrams to about 20 milligrams per day. In certain embodiments, the nanatinostat is administered at a dose of about 10 milligrams to about 40 milligrams per day. In certain embodiments, the nanatinostat is administered at a dose of about 10 milligrams per day. In certain embodiments, the nanatinostat is administered at a dose of about 20 milligrams per day. In certain embodiments, the nanatinostat is administered at a dose of about 40 milligrams per day. In certain embodiments, the nanatinostat is administered orally. In certain embodiments, the nanatinostat is administered b.i.d. In certain embodiments, the nanatinostat is administered q.d. In certain embodiments, the nucleoside analog is a substrate of a viral thymidine kinase. In certain embodiments, the nucleoside analog comprises valganciclovir, ganciclovir, acyclovir, valaciclovir, famciclovir, or a combination thereof. In certain embodiments, the nucleoside analog comprises valganciclovir. In certain embodiments, the valganciclovir is administered at dose of about 900 milligrams per day. In certain embodiments, the valganciclovir is administered at dose of about 450 milligrams per day. In certain embodiments, the checkpoint inhibitor antagonist is an antibody that binds to and inhibits the function of a checkpoint inhibitor. In certain embodiments, the checkpoint inhibitor comprises PD-1, PD-L1, PD-L2, or CTLA4. In certain embodiments, the checkpoint inhibitor comprises PD-1. In certain embodiments, the checkpoint inhibitor antagonist comprises nivolumab, pembrolizumab, cemiplimab, or a combination thereof. In certain embodiments, the checkpoint inhibitor comprises PD-L1. In certain embodiments, the checkpoint inhibitor antagonist comprises atezolizumab, avelumab, durvalumab, or a combination thereof. In certain embodiments, the checkpoint inhibitor comprises CTLA4. In certain embodiments, the checkpoint inhibitor antagonist comprises ipilimumab. In certain embodiments, the checkpoint inhibitor antagonist is administered intravenous. In certain embodiments, the checkpoint inhibitor antagonist is administered subcutaneous. In certain embodiments, the checkpoint inhibitor is administered at a dose of from about 1 milligram per kilogram to about 12 milligrams per kilogram. In certain embodiments, the checkpoint inhibitor is administered at a dose of about 2 milligrams per kilogram. In certain embodiments, the checkpoint inhibitor is administered at a dose of about 10 milligrams per kilogram. In certain embodiments, the checkpoint inhibitor is administered at a dose of about 200 milligrams. In certain embodiments, the checkpoint inhibitor is administered at a dose of about 400 milligrams. In certain embodiments, the checkpoint inhibitor is administered at a dose of about 300 milligrams. In certain embodiments, the checkpoint inhibitor is administered at a dose of about 240 milligrams. In certain embodiments, the checkpoint inhibitor is administered at a dose of about 350 milligrams. In certain embodiments, the checkpoint inhibitor is administered at a dose of about 480 milligrams. In certain embodiments, the checkpoint inhibitor is administered once every two weeks. In certain embodiments, the checkpoint inhibitor is administered once every three weeks. In certain embodiments, the checkpoint inhibitor is administered once every six weeks. In certain embodiments, the nanatinostat is administered on a weekly schedule and the individual is administered nanatinostat for 2 days of the weekly schedule. In certain embodiments, the nanatinostat is administered on a weekly schedule and the individual is administered the nanatinostat for 3 days of the weekly schedule. In certain embodiments, the nanatinostat is administered on a weekly schedule and the individual is administered the nanatinostat for 4 days of the weekly schedule. In certain embodiments, the one-week schedule is repeated one or more times. In certain embodiments, the cancer or tumor is a lymphoproliferative disorder. In certain embodiments, the lymphoproliferative disorder is B cell lymphoma or leukemia. In certain embodiments, the lymphoproliferative disorder is T cell lymphoma or leukemia. In certain embodiments, the lymphoproliferative disorder is a Hodgkin's lymphoma, a non-Hodgkin's lymphoma, or Burkitt's lymphoma. In certain embodiments, the cancer or tumor is a solid tumor. In certain embodiments, the cancer or tumor is a Herpesviridae associated cancer. In certain embodiments, the cancer or tumor is associated with human cytomegalovirus (CMV), Epstein-Barr virus (EBV), or herpes simplex virus 1 or 2 (HSV-1 or -2). In certain embodiments, the cancer or tumor is associated with Epstein-Barr virus (EBV). In certain embodiments, the cancer or tumor is an Epstein-Barr virus positive cancer or tumor. In certain embodiments, the cancer or tumor comprises a latent Epstein-Barr virus infection. In certain embodiments, the cancer or tumor is breast cancer, non-small cell lung cancer, melanoma, head and neck, lymphoepithelioma-like carcinoma, bladder, gastric cancer, nasopharyngeal carcinoma, leiomyosarcoma, or colorectal cancer. In certain embodiments, the cancer or tumor is nasopharyngeal carcinoma, leiomyosarcoma, lymphoepithelioma-like carcinoma, or gastric cancer. In certain embodiments, the cancer or tumor is nasopharyngeal carcinoma. In one aspect described herein is a method of treating an EBV-associated cancer or tumor in an individual, the method comprising administering to the individual afflicted with the cancer or tumor an effective amount of: a) nanatinostat; b) aciclovir, ganciclovir, valaciclovir, valganciclovir, or famciclovir; c) and a PD-1/PD-L1 axis inhibitor. In certain embodiments, the PD-1/PD-L1 axis inhibitor comprises nivolumab, pembrolizumab, cemiplimab, atezolizumab, avelumab, and durvalumab. In certain embodiments, the PD-1/PD-L1 axis inhibitor comprises nivolumab, pembrolizumab, or cemiplimab. In certain embodiments, the PD-1/PD-L1 axis inhibitor comprises pembrolizumab. In certain embodiments, the EBV-associated cancer is nasopharyngeal carcinoma, leiomyosarcoma, lymphoepithelioma-like carcinoma, or gastric cancer. In certain embodiments, the EBV-associated cancer is nasopharyngeal carcinoma.

### DETAILED DESCRIPTION

Described herein is a method of treating a cancer or tumor in an individual, the method comprising administering to the individual afflicted with the cancer or tumor an effective amount of: a) a histone deacetylase inhibitor (HDACi); b) a nucleoside analog; c) and a checkpoint inhibitor antagonist.

Described herein is a method of treating a cancer or tumor in an individual, the method comprising administering to the individual afflicted with the cancer or tumor an effective amount of: a) nanatinostat; b) a nucleoside analog; c) and a checkpoint inhibitor antagonist.

In the following description, certain specific details are set forth in order to provide a thorough understanding of various embodiments. However, one skilled in the art will understand that the embodiments provided may be practiced without these details. Unless the context requires otherwise, throughout the specification and claims which follow; the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein the term “about” refers to an amount that is near the stated amount by 10% or less.

As used herein the term “individual,” “patient,” or “subject” refers to individuals diagnosed with, suspected of being afflicted with, or at-risk of developing at least one disease for which the described compositions and method are useful for treating. In certain embodiments the individual is a mammal. In certain embodiments, the mammal is a mouse, rat, rabbit, dog, cat, horse, cow, sheep, pig, goat, llama, alpaca, or yak. In certain embodiments, the individual is a human.

Dosages may be referred to herein as QD, BID or TID. QD refers to dosing once a day. BID refers to dosing twice daily of the listed dose. TID refers to dosing three times a day of the listed dose. For example, 10 mg BID refers to two 10 mg dosage units deliver daily. BID doses may be spaced apart such that they are at least about 16, 12, 10, or 8 hours apart. TID doses may be spaced at about 4, 6, or 8-hour intervals.

The terms “administer,” “administering”, “administration,” and the like, as used herein, refer to the methods that are used to enable delivery of agents or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Administration techniques that in some instances are employed with the agents and methods described herein include, e.g., as discussed in Goodman and Gilman, The Pharmacological Basis of Therapeutics (current edition), Pergamon; and Remington's, Pharmaceutical Sciences (current edition), Mack Publishing Co., Easton, Pa. In certain embodiments, the agents and compositions described herein are administered orally. In some embodiments, the compositions described herein are administered parenterally.

The term “pharmaceutically acceptable” as used herein, refers to a material that does not abrogate the biological activity or properties of the agents described herein, and is relatively nontoxic (i.e., the toxicity of the material significantly outweighs the benefit of the material). In some instances, a pharmaceutically acceptable material is administered to an individual without causing significant undesirable biological effects or significantly interacting in a deleterious manner with any of the components of the composition in which it is contained.

The term “pharmaceutically acceptable excipient,” as used herein, refers to carriers and vehicles that are compatible with the active ingredient (for example, a compound of the invention) of a pharmaceutical composition of the invention (and preferably capable of stabilizing it) and not deleterious to the subject to be treated. For example, solubilizing agents that form specific, more soluble complexes with the compounds of the invention can be utilized as pharmaceutical excipients for delivery of the compounds. Suitable carriers and vehicles are known to those of extraordinary skill in the art. The term “excipient” as used herein will encompass all such carriers, adjuvants, diluents, solvents, or other inactive additives. Suitable pharmaceutically acceptable excipients include, but are not limited to, water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethyl-cellulose, polyvinylpyrrolidone, etc. The pharmaceutical compositions of the invention can also be sterilized and, if desired, mixed with auxiliary agents, e.g., lubricants, preservatives, stabilisers, wetting agents, emulsifiers, salts for influencing osmotic pressure, buffers, colorings, flavorings and/or aromatic substances and the like, which do not deleteriously react with the active compounds of the invention.

Among the provided antibodies are monoclonal antibodies, polyclonal antibodies, multispecific antibodies (for example, bispecific antibodies and polyreactive antibodies), and antibody fragments. The antibodies include antibody-conjugates and molecules comprising the antibodies, such as chimeric molecules. Thus, an antibody includes, but is not limited to, full- length and native antibodies, as well as fragments and portion thereof retaining the binding specificities thereof, such as any specific binding portion thereof including those having any number of, immunoglobulin classes and/or isotypes (e.g., IgG1, IgG2, IgG3, IgG4, IgM, IgA, IgD, IgE and IgM); and biologically relevant (antigen-binding) fragments or specific binding portions thereof, including but not limited to Fab, F (ab′)₂, Fv, and scFv (single chain or related entity). A monoclonal antibody is generally one within a composition of substantially homogeneous antibodies; thus, any individual antibodies comprised within the monoclonal antibody composition are identical except for possible naturally occurring mutations that may be present in minor amounts. A polyclonal antibody is a preparation that includes different antibodies of varying sequences that generally are directed against two or more different determinants (epitopes). The monoclonal antibody can comprise a human IgG1 constant region. The monoclonal antibody can comprise a human IgG4 constant region.

The term “antibody” herein is used in the broadest sense and includes polyclonal and monoclonal antibodies, including intact antibodies and functional (antigen-binding) antibody fragments thereof, including fragment antigen binding (Fab) fragments, F (ab′)₂ fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme. In certain embodiments the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs (See e.g., Kindt et al. Kuby Immunology, 6th ed., W.H. Freeman and Co., page 91(2007)). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively (See e.g., Portolano et al., J. Immunol. 150:880-887 (1993); Clarkson et al., Nature 352:624-628 (1991)).

Among the provided antibodies are antibody fragments. An “antibody fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; diabodies; linear antibodies; single-chain antibody molecules (e.g. scFv or sFv); and multispecific antibodies formed from antibody fragments. In particular embodiments, the antibodies are single-chain antibody fragments comprising a variable heavy chain region and/or a variable light chain region, such as scFvs.

The terms “polypeptide” and “protein” are used interchangeably to refer to a polymer of amino acid residues, and are not limited to a minimum length. Polypeptides, including the provided antibodies and antibody chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In some embodiments, amino acid sequence variants of the antibodies provided herein are contemplated. A variant typically differs from a polypeptide specifically disclosed herein in one or more substitutions, deletions, additions and/or insertions. Such variants can be naturally occurring or can be synthetically generated, for example, by modifying one or more of the above polypeptide sequences of the invention and evaluating one or more biological activities of the polypeptide as described herein and/or using any of a number of known techniques. For example, it may be desirable to improve the binding affinity and/or other biological properties of the antibody Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

The antibodies described herein can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, as of the filing date of this application.

### Therapeutic Methods

The methods described utilize a triple combination of a histone deacetylase inhibitor (HDACi), an anti-EBV or herpes antiviral (such as a nucleoside analog), and a checkpoint inhibitor antagonist, such as, for example, an antibody antagonist of the PD-1 or PD-L1 axis.

The methods described utilize a triple combination of nanatinostat, an anti-EBV or herpes antiviral (such as a nucleoside analog), and a checkpoint inhibitor antagonist, such as, for example, an antibody antagonist of the PD-1 or PD-L1 axis. The combination can comprise specific dosages and schedules. Therefor the methods contemplate embodiments, where the therapeutic agents are not administrated simultaneously, although an initial dose of any two or more of the three agents may be delivered on the same day, within a few days of each other, or substantially simultaneously. In other embodiments, an individual to be treated may be treated for one or more cycles with one or more of the agents as a single or double combination before addition of the second or third agent. Schedules referred to as weekly refer to treatment cycles that continue for 7 days before repeating if at all. Schedules that are recited as weekly do not begin on any particular day of the week. The treatment cycles may repeat 1, 2, 3, 4, 5, 6, 7, 8, 9, 10, 11, 12 or more times.

### Nanatinostat

As used herein nanatinostat is a class I HDAC inhibitor and is also referred to as CHR-3996 and VRx-3996, which are both chemically identical. The chemical formula of nanatinostat is 2-((1R,5S,6s)-6-(((6-fluoroquinolin-2-yl)methyl)amino)-3-azabicyclo[3.1.0]hexan-3-yl)-N-hydroxypyrimidine-5-carboxamide. Nanatinostat and methods of synthesis are disclosed in U.S. Pat. No. 7,932,246, which is incorporated by reference herein in its entirety.

Described herein are combinations with other HDAC inhibitors wherein the HDACi is selected from the list consisting of: vorinostat, romidepsin, mocetinostat, belinostat, pracinostat, givinostat, panobinostat, CUDC-101, zabinostat, chidamide, domatinostat, entinostat, and combinations thereof. In certain embodiments, the HDACi comprises vorinostat, romidepsin, mocetinostat, belinostat, pracinostat, givinostat, panobinostat, CUDC-101, zabinostat, chidamide, domatinostat, entinostat. In certain embodiments, the HDACi comprises vorinostat. In certain embodiments, the HDACi comprises romidepsin. In certain embodiments, the HDACi comprises mocetinostat. In certain embodiments, the HDACi comprises belinostat. In certain embodiments, the HDACi comprises pracinostat. In certain embodiments, the HDACi comprises givinostat. In certain embodiments the HDACi comprises panobinostat. In certain embodiments, the HDACi comprises CUDC-101. In certain embodiments, the HDACi comprises zabinostat. In certain embodiments, the HDACi comprises chidamide. In certain embodiments, the HDACi comprises domatinostat. In certain embodiments, the HDACi comprises entinostat.

In certain embodiments, an HDACi is administered at a total daily dose of about 5 milligrams to about 160 milligrams. In certain embodiments, the HDACi is administered at a total daily dose of about 5 milligrams to about 10 milligrams, about 5 milligrams to about 20 milligrams, about 5 milligrams to about 30 milligrams, about 5 milligrams to about 40 milligrams, about 5 milligrams to about 50 milligrams, about 5 milligrams to about 60 milligrams, about 5 milligrams to about 80 milligrams, about 5 milligrams to about 100 milligrams, about 5 milligrams to about 120 milligrams, about 5 milligrams to about 140 milligrams, about 5 milligrams to about 160 milligrams, about 10 milligrams to about 20 milligrams, about 10 milligrams to about 30 milligrams, about 10 milligrams to about 40 milligrams, about 10 milligrams to about 50 milligrams, about 10 milligrams to about 60 milligrams, about 10 milligrams to about 80 milligrams, about 10 milligrams to about 100 milligrams, about 10 milligrams to about 120 milligrams, about 10 milligrams to about 140 milligrams, about 10 milligrams to about 160 milligrams, about 20 milligrams to about 30 milligrams, about 20 milligrams to about 40 milligrams, about 20 milligrams to about 50 milligrams, about 20 milligrams to about 60 milligrams, about 20 milligrams to about 80 milligrams, about 20 milligrams to about 100 milligrams, about 20 milligrams to about 120 milligrams, about 20 milligrams to about 140 milligrams, about 20 milligrams to about 160 milligrams, about 30 milligrams to about 40 milligrams, about 30 milligrams to about 50 milligrams, about 30 milligrams to about 60 milligrams, about 30 milligrams to about 80 milligrams, about 30 milligrams to about 100 milligrams, about 30 milligrams to about 120 milligrams, about 30 milligrams to about 140 milligrams, about 30 milligrams to about 160 milligrams, about 40 milligrams to about 50 milligrams, about 40 milligrams to about 60 milligrams, about 40 milligrams to about 80 milligrams, about 40 milligrams to about 100 milligrams, about 40 milligrams to about 120 milligrams, about 40 milligrams to about 140 milligrams, about 40 milligrams to about 160 milligrams, about 50 milligrams to about 60 milligrams, about 50 milligrams to about 80 milligrams, about 50 milligrams to about 100 milligrams, about 50 milligrams to about 120 milligrams, about 50 milligrams to about 140 milligrams, about 50 milligrams to about 160 milligrams, about 60 milligrams to about 80 milligrams, about 60 milligrams to about 100 milligrams, about 60 milligrams to about 120 milligrams, about 60 milligrams to about 140 milligrams, about 60 milligrams to about 160 milligrams, about 80 milligrams to about 100 milligrams, about 80 milligrams to about 120 milligrams, about 80 milligrams to about 140 milligrams, about 80 milligrams to about 160 milligrams, about 100 milligrams to about 120 milligrams, about 100 milligrams to about 140 milligrams, about 100 milligrams to about 160 milligrams, about 120 milligrams to about 140 milligrams, about 120 milligrams to about 160 milligrams, or about 140 milligrams to about 160 milligrams. In certain embodiments, the HDACi is administered at a total daily dose of about 5 milligrams, about 10 milligrams, about 20 milligrams, about 30 milligrams, about 40 milligrams, about 50 milligrams, about 60 milligrams, about 80 milligrams, about 100 milligrams, about 120 milligrams, about 140 milligrams, or about 160 milligrams. In certain embodiments, the HDACi is administered at a total daily dose of at least about 5 milligrams, about 10 milligrams, about 20 milligrams, about 30 milligrams, about 40 milligrams, about 50 milligrams, about 60 milligrams, about 80 milligrams, about 100 milligrams, about 120 milligrams, or about 140 milligrams. In certain embodiments, the HDACi is administered at a total daily dose of at most about 10 milligrams, about 20 milligrams, about 30 milligrams, about 40 milligrams, about 50 milligrams, about 60 milligrams, about 80 milligrams, about 100 milligrams, about 120 milligrams, about 140 milligrams, or about 160 milligrams. This total daily dose can be split over two doses (b.i.d.), or three doses (t.i.d.) or administered in one dose (q.d.). An HDACi is suitably formulated for oral administration.

In certain embodiments, nanatinostat is administered at a total daily dose of about 5 milligrams to about 160 milligrams. In certain embodiments, nanatinostat is administered at a total daily dose of about 5 milligrams to about 10 milligrams, about 5 milligrams to about 20 milligrams, about 5 milligrams to about 30 milligrams, about 5 milligrams to about 40 milligrams, about 5 milligrams to about 50 milligrams, about 5 milligrams to about 60 milligrams, about 5 milligrams to about 80 milligrams, about 5 milligrams to about 100 milligrams, about 5 milligrams to about 120 milligrams, about 5 milligrams to about 140 milligrams, about 5 milligrams to about 160 milligrams, about 10 milligrams to about 20 milligrams, about 10 milligrams to about 30 milligrams, about 10 milligrams to about 40 milligrams, about 10 milligrams to about 50 milligrams, about 10 milligrams to about 60 milligrams, about 10 milligrams to about 80 milligrams, about 10 milligrams to about 100 milligrams, about 10 milligrams to about 120 milligrams, about 10 milligrams to about 140 milligrams, about 10 milligrams to about 160 milligrams, about 20 milligrams to about 30 milligrams, about 20 milligrams to about 40 milligrams, about 20 milligrams to about 50 milligrams, about 20 milligrams to about 60 milligrams, about 20 milligrams to about 80 milligrams, about 20 milligrams to about 100 milligrams, about 20 milligrams to about 120 milligrams, about 20 milligrams to about 140 milligrams, about 20 milligrams to about 160 milligrams, about 30 milligrams to about 40 milligrams, about 30 milligrams to about 50 milligrams, about 30 milligrams to about 60 milligrams, about 30 milligrams to about 80 milligrams, about 30 milligrams to about 100 milligrams, about 30 milligrams to about 120 milligrams, about 30 milligrams to about 140 milligrams, about 30 milligrams to about 160 milligrams, about 40 milligrams to about 50 milligrams, about 40 milligrams to about 60 milligrams, about 40 milligrams to about 80 milligrams, about 40 milligrams to about 100 milligrams, about 40 milligrams to about 120 milligrams, about 40 milligrams to about 140 milligrams, about 40 milligrams to about 160 milligrams, about 50 milligrams to about 60 milligrams, about 50 milligrams to about 80 milligrams, about 50 milligrams to about 100 milligrams, about 50 milligrams to about 120 milligrams, about 50 milligrams to about 140 milligrams, about 50 milligrams to about 160 milligrams, about 60 milligrams to about 80 milligrams, about 60 milligrams to about 100 milligrams, about 60 milligrams to about 120 milligrams, about 60 milligrams to about 140 milligrams, about 60 milligrams to about 160 milligrams, about 80 milligrams to about 100 milligrams, about 80 milligrams to about 120 milligrams, about 80 milligrams to about 140 milligrams, about 80 milligrams to about 160 milligrams, about 100 milligrams to about 120 milligrams, about 100 milligrams to about 140 milligrams, about 100 milligrams to about 160 milligrams, about 120 milligrams to about 140 milligrams, about 120 milligrams to about 160 milligrams, or about 140 milligrams to about 160 milligrams. In certain embodiments, nanatinostat is administered at a total daily dose of about 5 milligrams, about 10 milligrams, about 20 milligrams, about 30 milligrams, about 40 milligrams, about 50 milligrams, about 60 milligrams, about 80 milligrams, about 100 milligrams, about 120 milligrams, about 140 milligrams, or about 160 milligrams. In certain embodiments, nanatinostat is administered at a total daily dose of at least about 5 milligrams, about 10 milligrams, about 20 milligrams, about 30 milligrams, about 40 milligrams, about 50 milligrams, about 60 milligrams, about 80 milligrams, about 100 milligrams, about 120 milligrams, or about 140 milligrams. In certain embodiments, nanatinostat is administered at a total daily dose of at most about 10 milligrams, about 20 milligrams, about 30 milligrams, about 40 milligrams, about 50 milligrams, about 60 milligrams, about 80 milligrams, about 100 milligrams, about 120 milligrams, about 140 milligrams, or about 160 milligrams. This total daily dose can be split over two doses (b.i.d.), or three doses (t.i.d.) or administered in one dose (q.d.). Nanatinostat is suitably formulated for oral administration.

### Nucleoside Analogs

Nucleoside analogs are agents which contain a nucleic acid analogue and a sugar. Nucleic acid analogs are substances that are analogous to naturally occurring nucleic acids. Guanosine analogs are generally useful in the methods described herein. Such useful guanosine analogs comprise those that are phosphorylated by a viral thymidine or protein kinase including valganciclovir, ganciclovir, acyclovir, valaciclovir, or famciclovir. In certain embodiments, the nucleoside analog to be included in the methods described herein comprise valganciclovir, ganciclovir, acyclovir, valaciclovir, or famciclovir, or a combination thereof. In certain embodiments, the nucleoside analog to be included in the methods described herein comprises valganciclovir. In certain embodiments, the nucleoside analog to be included in the methods described herein comprises ganciclovir. In certain embodiments, the nucleoside analog to be included in the methods described herein comprises acyclovir. In certain embodiments, the nucleoside analog to be included in the methods described herein comprises famciclovir. In certain embodiments, the nucleoside analog to be included in the methods described herein comprises valaciclovir.

In some embodiments, the antiviral agent is administered at a dose of less than 3000 mg/day. In some embodiments, the antiviral agent is administered at a dose of about 10 mg/day, about 20 mg/day, about 50 mg/day, about 100 mg/day, about 150 mg/day, about 200 mg/day, about 250 mg/day, about 300 mg/day, about 350 mg/day, about 400 mg/day, about 450 mg/day, about 500 mg/day, about 600 mg/day, about 700 mg/day, about 800 mg/day, about 900 mg/day, about 1000 mg/day, about 1200 mg/day, about 1250 mg/day, about 1400 mg/day, about 1500 mg/day, about 1600 mg/day, about 1750 mg/day, about 1800 mg/day, about 1900 mg/day, about 2000 mg/day, about 2250 mg/day, about 2500 mg/day, about 2750 mg/day, about 3000 mg/day, about 3250 mg/day, about 3500 mg/day, about 3750 mg/day, about 4000 mg/day, about 4250 mg/day, about 4500 mg/day, about 4750 mg/day, or about 5000 mg/day. In certain embodiments, the antiviral agent is administered at a dose of less than 10 mg/day, less than 20 mg/day, less than 50 mg/day, less than 100 mg/day, less than 150 mg/day, less than 200 mg/day, less than 250 mg/day, less than 300 mg/day, less than 350 mg/day, less than 400 mg/day, less than 450 mg/day, less than 500 mg/day, less than 600 mg/day, less than 700 mg/day, less than 800 mg/day, less than 900 mg/day, less than 1000 mg/day, less than 1200 mg/day, less than 1250 mg/day, less than 1400 mg/day, less than 1500 mg/day, less than 1600 mg/day, less than 1750 mg/day, less than 1800 mg/day, less than 1900 mg/day, less than 2000 mg/day, less than 2250 mg/day, less than 2500 mg/day, less than 2750 mg/day, less than 3000 mg/day, less than 3250 mg/day, less than 3500 mg/day, less than 3750 mg/day, less than 4000 mg/day, less than 4250 mg/day, less than 4500 mg/day, less than 4750 mg/day, or less than 5000 mg/day. In some embodiments, the antiviral agent is administered at a dose of more than 10 mg/day, more than 20 mg/day, more than 50 mg/day, more than 100 mg/day, more than 150 mg/day, more than 200 mg/day, more than 250 mg/day, more than 300 mg/day, more than 350 mg/day, more than 400 mg/day, more than 450 mg/day, more than 500 mg/day, more than 600 mg/day, more than 700 mg/day, more than 800 mg/day, more than 900 mg/day, more than 1000 mg/day, more than 1200 mg/day, more than 1250 mg/day, more than 1400 mg/day, more than 1500 mg/day, more than 1600 mg/day, more than 1750 mg/day, more than 1800 mg/day, more than 1900 mg/day, more than 2000 mg/day, more than 2250 mg/day, more than 2500 mg/day, more than 2750 mg/day, more than 3000 mg/day, more than 3250 mg/day, more than 3500 mg/day, more than 3750 mg/day, more than 4000 mg/day, more than 4250 mg/day, more than 4500 mg/day, more than 4750 mg/day, or more than 5000 mg/day. In certain embodiments, the antiviral agent is administered at a dose of more than 10 mg/day and less than 5000 mg/day. In some embodiments, the antiviral agent is administered at a dose of more than 200 mg/day and less than 1000 mg/day. In certain embodiments, the antiviral agent is administered once a day (q.d. QD.), twice a day (b.i.d., BID), or thrice a day (t.i.d., TID). In some embodiments, the antiviral agent is administered daily, once a week, twice a week, three times a week, four times a week, or five times a week.

In certain embodiments, the nucleoside analog comprises valganciclovir and is administered daily at dose of 1,800, 900, or 450 milligrams. In certain embodiments, the nucleoside analog comprises valganciclovir and is administered daily at dose of 1,800 milligrams. In certain embodiments, the nucleoside analog comprises valganciclovir and is administered daily at dose of 900 milligrams.

### Checkpoint Inhibitor Antagonists

Checkpoint molecules are expressed by immune cells in the adaptive immune system and provide feedback, both positive and negative on the developing immune response to an antigen. Checkpoint inhibitors provide negative feedback and functionally decrease immune responses. Key checkpoint inhibitors that reduce immune responses to given antigens include, but are not limited to, PD-1, PD-L1, PD-L2, and CTLA4. For example, and not to be bound by theory, PD-1 (usually expressed on T cells) engages (PDL-1 and/or PD-L2) and promotes immunosuppressive intracellular signaling that reduces T cell activity, and together this interaction is the PD-1/PD-L1 axis. This axis is important as inhibiting one arm, either PD-1 or PDL-1 or PD-L2 inhibits negative signaling through this axis. Many tumor cells acquire expression of PD-L1 or PD-L2 contributing to immune evasion by tumors.

In certain embodiments, the checkpoint inhibitor antagonist is an antibody that binds to and inhibits the function of a checkpoint inhibitor. In certain embodiments, the checkpoint inhibitor comprises PD-1, PD-L1, PD-L2, or CTLA4. In certain embodiments, the checkpoint inhibitor comprises PD-1. In certain embodiments, the checkpoint inhibitor antagonist comprises nivolumab, pembrolizumab, or cemiplimab. In certain embodiments, the checkpoint inhibitor comprises PD-L1. In certain embodiments, the checkpoint inhibitor antagonist comprises atezolizumab, avelumab, or durvalumab. In certain embodiments, the checkpoint inhibitor comprises CTLA4. In certain embodiments, the checkpoint inhibitor antagonist comprises ipilimumab.

As the active binding agent of the antibodies listed herein comprise CDR sequences also envisioned are checkpoint inhibitor antagonistic antibodies that include the CDR regions from any one or more of nivolumab, pembrolizumab, cemiplimab atezolizumab, avelumab, durvalumab, or ipilimumab. In certain embodiments, the antibodies comprise a heavy chain CDR1, heavy chain CDR2, heavy chain CDR3, light chain CDR1, light chain CDR2, and/or light chain CDR3.

In certain embodiments, the checkpoint inhibitor antagonist antibody is administered at dose of about 200 milligrams to about 600 milligrams every three weeks. In certain embodiments, the checkpoint inhibitor antagonist antibody is administered at dose of about 200 milligrams to about 300 milligrams, about 200 milligrams to about 400 milligrams, about 200 milligrams to about 500 milligrams, about 200 milligrams to about 600 milligrams, about 300 milligrams to about 400 milligrams, about 300 milligrams to about 500 milligrams, about 300 milligrams to about 600 milligrams, about 400 milligrams to about 500 milligrams, about 400 milligrams to about 600 milligrams, or about 500 milligrams to about 600 milligrams every three weeks. In certain embodiments, the checkpoint inhibitor antagonist antibody is administered at dose of about 200 milligrams, about 300 milligrams, about 400 milligrams, about 500 milligrams, or about 600 milligrams every three weeks. In certain embodiments, the checkpoint inhibitor antagonist antibody is administered at dose of about at least about 200 milligrams, about 300 milligrams, about 400 milligrams, or about 500 milligrams every three weeks. In certain embodiments, the checkpoint inhibitor antagonist antibody is administered at dose of about at most about 300 milligrams, about 400 milligrams, about 500 milligrams, or about 600 milligrams every three weeks.

In certain embodiments, the checkpoint inhibitor antagonist antibody is administered at dose of about 200 milligrams to about 600 milligrams every six weeks. In certain embodiments, the checkpoint inhibitor antagonist antibody is administered at dose of about 200 milligrams to about 300 milligrams, about 200 milligrams to about 400 milligrams, about 200 milligrams to about 500 milligrams, about 200 milligrams to about 600 milligrams, about 300 milligrams to about 400 milligrams, about 300 milligrams to about 500 milligrams, about 300 milligrams to about 600 milligrams, about 400 milligrams to about 500 milligrams, about 400 milligrams to about 600 milligrams, or about 500 milligrams to about 600 milligrams every six weeks. In certain embodiments, the checkpoint inhibitor antagonist antibody is administered at dose of about 200 milligrams, about 300 milligrams, about 400 milligrams, about 500 milligrams, or about 600 milligrams every six weeks. In certain embodiments, the checkpoint inhibitor antagonist antibody is administered at dose of about at least about 200 milligrams, about 300 milligrams, about 400 milligrams, or about 500 milligrams every six weeks. In certain embodiments, the checkpoint inhibitor antagonist antibody is administered at dose of about at most about 300 milligrams, about 400 milligrams, about 500 milligrams, or about 600 milligrams every six weeks.

In certain embodiments, the checkpoint inhibitor antagonist antibody is administered at dose of about 240 milligrams every 2 weeks.

In certain embodiments, the checkpoint inhibitor antagonist antibody is nivolumab and is administered at dose of about 240 milligrams every 2 weeks.

In certain embodiments, the checkpoint inhibitor antagonist antibody is administered at dose of about 480 milligrams every 4 weeks.

In certain embodiments, the checkpoint inhibitor antagonist antibody is nivolumab and is administered at dose of about 480 milligrams every 4 weeks.

In certain embodiments, the checkpoint inhibitor antagonist antibody is administered at dose of about 200 milligrams every 3 weeks.

In certain embodiments, the checkpoint inhibitor antagonist antibody is pembrolizumab and is administered at dose of about 200 milligrams every 3 weeks.

In certain embodiments, the checkpoint inhibitor antagonist antibody is administered at dose of about 400 milligrams every 6 weeks.

In certain embodiments, the checkpoint inhibitor antagonist antibody is pembrolizumab and is administered at dose of about 400 milligrams every 6 weeks.

In certain embodiments, the checkpoint inhibitor antagonist antibody is administered at dose of about 350 milligrams every 3 weeks.

In certain embodiments, the checkpoint inhibitor antagonist antibody is cemiplimaband is administered at dose of about 350 milligrams every 3 weeks.

Treatment with checkpoint inhibitors can be initiated simultaneously or substantially simultaneously with the initiation of nanatinostat and nucleoside analog. In some cases nanatinostat and nucleoside analog can be added to a course of treatment with a checkpoint inhibitor antagonist that is ongoing, or checkpoint inhibitor antagonist can be added to a course of treatment with nanatinostat and nucleoside that is ongoing. The checkpoint inhibitor antagonist may be administered after about 1 week of beginning treatment with nanatinostat and nucleoside analog. The checkpoint inhibitor antagonist may be administered after about 2 weeks of beginning treatment with nanatinostat and nucleoside analog. The checkpoint inhibitor antagonist may be administered after about 3 weeks of beginning treatment with nanatinostat and nucleoside analog. The checkpoint inhibitor antagonist may be administered after about 4 weeks of beginning treatment with nanatinostat and nucleoside analog.

The checkpoint inhibitor antagonist may be administered after about 1 week of beginning treatment with nanatinostat and valganciclovir. The checkpoint inhibitor antagonist may be administered after about 2 weeks of beginning treatment with nanatinostat and valganciclovir. The checkpoint inhibitor antagonist may be administered after about 3 weeks of beginning treatment with nanatinostat and valganciclovir. The checkpoint inhibitor antagonist may be administered after about 4 weeks of beginning treatment with nanatinostat and valganciclovir.

Checkpoint inhibitor antagonists may suitably be administered intravenously. Checkpoint inhibitor antagonists may suitably administered subcutaneously.

### Combination Schedules

While the checkpoint inhibitor agonists have very low toxicity generally and can be dosed at levels at or near previously approved doses, the same is not necessarily true of the combination of nanatinostat and nucleoside analog. HDAC inhibitors are associated with some toxicity and therefore may need to be dosed intermittently as opposed to daily while the nucleoside analog such as valganciclovir can be dosed daily at 900 milligrams normally or 450 milligrams if kidney or liver toxicity is indicated.

In certain embodiments, nanatinostat is dosed 5 milligrams daily on a weekly schedule with nanatinostat dosed on one day. In certain embodiments, nanatinostat is dosed 5 milligrams daily on a weekly schedule with nanatinostat dosed on two days. In certain embodiments, nanatinostat is dosed 5 milligrams daily on a weekly schedule with nanatinostat dosed on three days. In certain embodiments, nanatinostat is dosed 5 milligrams daily on a weekly schedule with nanatinostat dosed on four days. In certain embodiments the days are not consecutive.

In certain embodiments, nanatinostat is dosed 10 milligrams daily on a weekly schedule with nanatinostat dosed on one day. In certain embodiments, nanatinostat is dosed 10 milligrams daily on a weekly schedule with nanatinostat dosed on two days. In certain embodiments, nanatinostat is dosed 10 milligrams daily on a weekly schedule with nanatinostat dosed on three days. In certain embodiments, nanatinostat is dosed 10 milligrams daily on a weekly schedule with nanatinostat dosed on four days. In certain embodiments the days are not consecutive.

In certain embodiments, nanatinostat is dosed 20 milligrams daily on a weekly schedule with nanatinostat dosed on one day. In certain embodiments, nanatinostat is dosed 20 milligrams daily on a weekly schedule with nanatinostat dosed on two days. In certain embodiments, nanatinostat is dosed 20 milligrams daily on a weekly schedule with nanatinostat dosed on three days. In certain embodiments, nanatinostat is dosed 20 milligrams daily on a weekly schedule with nanatinostat dosed on four days. In certain embodiments the days are not consecutive.

In certain embodiments, nanatinostat is dosed 30 milligrams daily on a weekly schedule with nanatinostat dosed on one day. In certain embodiments, nanatinostat is dosed 30 milligrams daily on a weekly schedule with nanatinostat dosed on two days. In certain embodiments, nanatinostat is dosed 30 milligrams daily on a weekly schedule with nanatinostat dosed on three days. In certain embodiments, nanatinostat is dosed 30 milligrams daily on a weekly schedule with nanatinostat dosed on four days. In certain embodiments the days are not consecutive.

In certain embodiments, nanatinostat is dosed 40 milligrams daily on a weekly schedule with nanatinostat dosed on one day. In certain embodiments, nanatinostat is dosed 40 milligrams daily on a weekly schedule with nanatinostat dosed on two days. In certain embodiments, nanatinostat is dosed 40 milligrams daily on a weekly schedule with nanatinostat dosed on three days. In certain embodiments, nanatinostat is dosed 40 milligrams daily on a weekly schedule with nanatinostat dosed on four days. In certain embodiments the days are not consecutive.

In certain embodiments, nanatinostat is dosed 50 milligrams daily on a weekly schedule with nanatinostat dosed on one day. In certain embodiments, nanatinostat is dosed 50 milligrams daily on a weekly schedule with nanatinostat dosed on two days. In certain embodiments, nanatinostat is dosed 50 milligrams daily on a weekly schedule with nanatinostat dosed on three days. In certain embodiments, nanatinostat is dosed 50 milligrams daily on a weekly schedule with nanatinostat dosed on four days. In certain embodiments the days are not consecutive.

In certain embodiments, nanatinostat is dosed 60 milligrams daily on a weekly schedule with nanatinostat dosed on one day. In certain embodiments, nanatinostat is dosed 60 milligrams daily on a weekly schedule with nanatinostat dosed on two days. In certain embodiments, nanatinostat is dosed 60 milligrams daily on a weekly schedule with nanatinostat dosed on three days. In certain embodiments, nanatinostat is dosed 60 milligrams daily on a weekly schedule with nanatinostat dosed on four days. In certain embodiments the days are not consecutive.

In certain embodiments, nanatinostat is dosed 80 milligrams daily on a weekly schedule with nanatinostat dosed on one day. In certain embodiments, nanatinostat is dosed 80 milligrams daily on a weekly schedule with nanatinostat dosed on two days. In certain embodiments, nanatinostat is dosed 80 milligrams daily on a weekly schedule with nanatinostat dosed on three days. In certain embodiments, nanatinostat is dosed 80 milligrams daily on a weekly schedule with nanatinostat dosed on four days. In certain embodiments the days are not consecutive.

Doses for intermittent dosing may be separated by one day with no nanatinostat dosing. Doses for intermittent dosing may be separated by two days with no nanatinostat dosing. Doses for intermittent dosing may be separated by three days with no nanatinostat dosing. Doses for intermittent dosing may be separated by four days with no nanatinostat dosing.

When dosed twice a week the doses may be on days 1 and 3, 1 and 4, 1 and 5, or l and 6. When dosed twice a week the doses may be on days 2 and 4, 2 and 5, or 2 and 6.

The schedules described herein can also be administered to certain patients with nanatinostat or nucleoside analogue side effects. In certain embodiments, the methods described herein encompass selecting a patent with thrombocytopenia. In certain embodiments, the methods and nanatinostat compositions described herein are for use in a patent with thrombocytopenia. Thrombocytopenia is generally defined as a platelet count below 150,000 platelets per microliter. In certain embodiments, the patient can be selected for treatment by the methods herein with a platelet count of below about 50,000; 75,000; 100,000, or 125,000 platelets per microliter. In certain embodiments, the methods do not encompass a set schedule of nanatinostat administration, but further monitoring for resolution of thrombocytopenia before retreatment with nanatinostat. In certain embodiments, if a patient has one or more dose interruptions for nanatinostat toxicity or has had to interrupt for more than 14 days, the methods described herein include a dose reduction for the nanatinostat inhibitor. In certain embodiments with a dose reduction, the nanatinostat will comprise nanatinostat and the dose reduction will be in 5 mg increments. In certain embodiments, the nanatinostat dose is re-escalated.

### Therapeutic Indications

In certain embodiments, disclosed herein, are methods and combinations useful for the treatment of a cancers or tumors. Treatment refers to a method that seeks to improve or ameliorate the condition being treated. With respect to cancer, treatment includes, but is not limited to, reduction of tumor volume, reduction in growth of tumor volume, increase in progression-free survival, or overall life expectancy. In certain embodiments, treatment will effect remission of a cancer being treated. In certain embodiments, treatment encompasses use as a prophylactic or maintenance dose intended to prevent reoccurrence or progression of a previously treated cancer or tumor. It is understood by those of skill in the art that not all individuals will respond equally or at all to a treatment that is administered, nevertheless these individuals are considered to be treated.

Epstein-Barr virus (EBV), a member of the y-herpesvirus family, was the first virus directly implicated in the development of a human tumor and is formally classified as a carcinogenic agent by the World Health Organization (WHO). Primary infection with EBV typically occurs in childhood and is generally asymptomatic; however, infection later in life may manifest as infectious mononucleosis. Once infected, individuals remain lifelong carriers of the virus, with >90% of the world's population asymptomatically infected with EBV.

Latent infection and intermittent reactivation are 2 important characteristics of the EBV lifecycle. The maintenance of latent EBV infection requires the expression of a small subset of genes. Specific expression patterns (Types I-III) of these genes are associated with specific EBV-driven malignancies. EBV is associated with a wide spectrum of lymphoid malignancies, including B-cell, T/natural killer (NK)-cell and Hodgkin lymphomas, and solid tumors such as nasopharyngeal carcinoma (NPC), gastric cancer (and other lymphoepithelioma-like cancers involving the lung and other organs), and leiomyosarcomas.

In certain embodiments, the cancer or tumor is a solid cancer or tumor. In certain embodiments, the cancer or tumor is a blood cancer or tumor. In certain embodiments, the cancer or tumor comprises breast, heart, lung, small intestine, colon, spleen, kidney, bladder, head and neck, ovarian, prostate, brain, pancreatic, skin, bone, bone marrow, blood, thymus, uterine, testicular, and liver tumors. In certain embodiments, the cancer or tumor is associated or positive for a member of the Herpesviridae. In certain embodiments, the cancer is one associated with cytomegalovirus, HHV-8, HSV-1, HSV-2, or Epstein-Barr Virus (EBV). In certain embodiments, the EBV associated or positive cancer or tumor comprises Hodgkin's lymphoma, non-Hodgkin's lymphoma, Burkitt's lymphoma, or Nasopharyngeal carcinoma. In certain embodiments, the EBV associated cancer is Nasopharyngeal carcinoma. In certain embodiments, the EBV associated or positive cancer or tumor is nasopharyngeal carcinoma, leiomyosarcoma, lymphoepithelioma-like carcinoma, or gastric cancer.

In certain embodiments, the antibodies can be administered to a subject in need thereof by any route suitable for the administration of antibody-containing pharmaceutical compositions, such as, for example, subcutaneous, intraperitoneal, intravenous, intramuscular, intratumoral, or intracerebral, etc. In certain embodiments, the antibodies are administered intravenously. In certain embodiments, the antibodies are administered subcutaneously. In certain embodiments, the antibodies are administered intratumoral. In certain embodiments, the antibodies are administered on a suitable dosage schedule, for example, weekly, twice weekly, monthly, twice monthly, once every two weeks, once every three weeks, or once a month etc. In certain embodiments, the antibodies are administered once every three weeks. The antibodies can be administered in any therapeutically effective amount. In certain embodiments, the therapeutically acceptable amount is between about 0.1 mg/kg and about 50 mg/kg. In certain embodiments, the therapeutically acceptable amount is between about 1 mg/kg and about 40 mg/kg. In certain embodiments, the therapeutically acceptable amount is between about 5 mg/kg and about 30 mg/kg. Therapeutically effective amounts include amounts are those sufficient to ameliorate one or more symptoms associated with the disease or affliction to be treated.

### Pharmaceutically Acceptable Excipients, Carriers, and Diluents

In certain embodiments the anti-checkpoint inhibitor antibodies of the current disclosure are included in a pharmaceutical composition comprising one or more pharmaceutically acceptable excipients, carriers, and diluents. In certain embodiments, the antibodies of the current disclosure are administered suspended in a sterile solution. In certain embodiments, the solution comprises about 0.9% NaCl. In certain embodiments, the solution comprises about 5.0% dextrose. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EDTA or EGTA.

In certain embodiments, the antibodies of the current disclosure are shipped/stored lyophilized and reconstituted before administration. In certain embodiments, lyophilized antibody formulations comprise a bulking agent such as, mannitol, sorbitol, sucrose, trehalose, dextran 40, or combinations thereof. The lyophilized formulation can be contained in a vial comprised of glass or other suitable non-reactive material. The antibodies when formulated, whether reconstituted or not, can be buffered at a certain pH, generally less than 7.0. In certain embodiments, the pH can be between 4.5 and 6.5, 4.5 and 6.0, 4.5 and 5.5, 4.5 and 5.0, or 5.0 and 6.0.

For oral administration, the one or one or more agents can be formulated readily by combining the one or more active agents with pharmaceutically acceptable carriers well known in the art. Such carriers can enable the one or more agents to be formulated as tablets, including chewable tablets, pills, dragees, capsules, lozenges, hard candy, liquids, gels, syrups, slurries, powders, suspensions, elixirs, wafers, and the like, for oral ingestion by a patient to be treated. Such formulations can comprise pharmaceutically acceptable carriers including solid diluents or fillers, sterile aqueous media and various non-toxic organic solvents. Generally, the agents of the invention can be included at concentration levels ranging from about 0.5%, about 5%, about 10%, about 20%, or about 30% to about 50%, about 60%, about 70%, about 80% or about 90% by weight of the total composition of oral dosage forms, in an amount sufficient to provide a desired unit of dosage.

Aqueous suspensions for oral use can contain one or more agents with pharmaceutically acceptable excipients, such as a suspending agent (e.g., methyl cellulose), a wetting agent (e.g., lecithin, lysolecithin and/or a long-chain fatty alcohol), as well as coloring agents, preservatives, flavoring agents, and the like.

Oils or non-aqueous solvents can be required to bring one or more agents into solution, due to, for example, the presence of large lipophilic moieties. Alternatively, emulsions, suspensions, or other preparations, for example, liposomal preparations, can be used. With respect to liposomal preparations, any known methods for preparing liposomes for treatment of a condition can be used. See, for example, Bangham et al., J. Mol. Biol. 23: 238-252 (1965) and Szoka et al., Proc. Natl Acad. Sci. USA 75: 4194-4198 (1978), incorporated herein by reference. Ligands can also be attached to the liposomes to direct these compositions to particular sites of action. One or more agents can also be integrated into foodstuffs, e.g, cream cheese, butter, salad dressing, or ice cream to facilitate solubilization, administration, and/or compliance in certain patient populations.

Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; flavoring elements, cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl pyrrolidone (PVP). Disintegrating agents can be added, for example, the cross-linked polyvinyl pyrrolidone, agar, or alginic acid or a salt thereof such as sodium alginate. One or more agents can also be formulated as a sustained release preparation.

Dragee cores can be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of one or more active agents.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active agents can be dissolved or suspended in suitable liquids, such as fatty oils, liquid paraffin, or liquid polyethylene glycols. In addition, stabilizers can be added. All formulations for oral administration can be in dosages suitable for administration.

Also described herein are kits comprising one or more of the agents described herein in a suitable container and one or more additional components selected from: instructions for use; a diluent, an excipient, a carrier, and a device for administration. In certain embodiments, a kit comprises nanatinostat, a nucleoside analog, and checkpoint inhibitor antagonist in suitable packaging.

### Specific Embodiments of the Invention

1. A method of treating a cancer or tumor in an individual, the method comprising administering to the individual afflicted with the cancer or tumor an effective amount of: a) a histone deacetylase inhibitor (HDACi); b) a nucleoside analog; c) and a checkpoint inhibitor antagonist.
2. The method of embodiment 1, wherein the HDACi comprises vorinostat, romidepsin, mocetinostat, belinostat, pracinostat, givinostat, panobinostat, CUDC-101, zabinostat, chidamide, domatinostat, entinostat, and combinations thereof.
3. The method of embodiment 1 wherein the HDACi comprises nanatinostat.
4. The method of any one of embodiments 1 to 3, wherein the nanatinostat is administered at a dose of about 5 milligrams to about 160 milligrams per day.
5. The method of any one of embodiments 1 to 3, wherein the nanatinostat is administered at a dose of about 5 milligrams to about 80 milligrams per day.
6. The method of any one of embodiments 1 to 3, wherein the nanatinostat is administered at a dose of about 5 milligrams to about 40 milligrams per day.
7. The method of any one of embodiments 1 to 3, wherein the nanatinostat is administered at a dose of about 5 milligrams to about 20 milligrams per day.
8. The method of any one of embodiments 1 to 3, wherein the nanatinostat is administered at a dose of about 10 milligrams to about 20 milligrams per day.
9. The method of any one of embodiments 1 to 3, wherein the nanatinostat is administered at a dose of about 10 milligrams to about 40 milligrams per day.
10. The method of any one of embodiments 1 to 3, wherein the nanatinostat is administered at a dose of about 10 milligrams per day.
11. The method of any one of embodiments 1 to 3, wherein the nanatinostat is administered at a dose of about 20 milligrams per day.
12. The method of any one of embodiments 1 to 3, wherein the nanatinostat is administered at a dose of about 40 milligrams per day.
13. The method of any one of embodiments 1 to 12, wherein the nanatinostat is administered orally.
14. The method of any one of embodiments 1 to 13, wherein the nanatinostat is administered b.i.d.
15. The method of any one of embodiments 1 to 13, wherein the nanatinostat is administered q.d.
16. The method of any one of embodiments 1 to 15, wherein the nucleoside analog is a substrate of a viral thymidine kinase.
17. The method of any one of embodiments 1 to 16, wherein the nucleoside analog comprises valganciclovir, ganciclovir, acyclovir, valaciclovir, famciclovir or a combination thereof.
18. The method of any one of embodiments 1 to 16, wherein the nucleoside analog comprises valganciclovir.
19. The method of embodiment 18, wherein the valganciclovir is administered at dose of about 900 milligrams per day.
20. The method of embodiment 18, wherein the valganciclovir is administered at dose of about 450 milligrams per day.
21. The method of any one of embodiments 1 to 20, wherein the checkpoint inhibitor antagonist is an antibody that binds to and inhibits the function of a checkpoint inhibitor.
22. The method of any one of embodiments 1 to 21, wherein the checkpoint inhibitor comprises PD-1, PD-L1, PD-L2, or CTLA4.
23. The method of embodiment 22, wherein the checkpoint inhibitor comprises PD-1.
24. The method of embodiment 23, wherein the checkpoint inhibitor antagonist comprises nivolumab, pembrolizumab, cemiplimab, or a combination thereof.
25. The method of embodiment 22, wherein the checkpoint inhibitor comprises PD-L1.
26. The method of embodiment 25, wherein the checkpoint inhibitor antagonist comprises atezolizumab, avelumab, durvalumab, or a combination thereof.
27. The method of embodiment 22, wherein the checkpoint inhibitor comprises CTLA4.
28. The method of embodiment 27, wherein the checkpoint inhibitor antagonist comprises ipilimumab.
29. The method of any one of embodiments 1 to 28, wherein the checkpoint inhibitor antagonist is administered intravenous.
30. The method of any one of embodiments 1 to 28, wherein the checkpoint inhibitor antagonist is administered subcutaneous.
31. The method of any one of embodiments 1 to 30, wherein the checkpoint inhibitor is administered at a dose of about 200 milligrams.
32. The method of any one of embodiments 1 to 30, wherein the checkpoint inhibitor is administered at a dose of about 400 milligrams.
33. The method of any one of embodiments 1 to 30, wherein the checkpoint inhibitor is administered at a dose of from about 1 milligram per kilogram to about 12 milligrams per kilogram.
34. The method of any one of embodiments 1 to 30, wherein the checkpoint inhibitor is administered at a dose of about 2 milligrams per kilogram.
35. The method of any one of embodiments 1 to 30, wherein the checkpoint inhibitor is administered at a dose of about 10 milligrams per kilogram.
36. The method of any one of embodiments 1 to 30, wherein the checkpoint inhibitor is administered at a dose of about 300 milligrams.
37. The method of any one of embodiments 1 to 30, wherein the checkpoint inhibitor is administered at a dose of about 240 milligrams.
38. The method of any one of embodiments 1 to 30, wherein the checkpoint inhibitor is administered at a dose of about 350 milligrams.
39. The method of any one of embodiments 1 to 30, wherein the checkpoint inhibitor is administered at a dose of about 480 milligrams.
40. The method of any one of embodiments 1 to 39, wherein the checkpoint inhibitor is administered once every two weeks.
41. The method of any one of embodiments 1 to 39, wherein the checkpoint inhibitor is administered once every three weeks.
42. The method of any one of embodiments 1 to 39, wherein the checkpoint inhibitor is administered once every four weeks.
43. The method of any one of embodiments 1 to 39, wherein the checkpoint inhibitor is administered once every six weeks.
44. The method of any one of embodiments 1 to 43, wherein the nanatinostat is administered on a weekly schedule and the individual is administered nanatinostat for 2 days of the weekly schedule.
45. The method of any one of embodiments 1 to 43, wherein the nanatinostat is administered on a weekly schedule and the individual is administered the nanatinostat for 3 days of the weekly schedule.
46. The method of any one of embodiments 1 to 43, wherein the nanatinostat is administered on a weekly schedule and the individual is administered the nanatinostat for 4 days of the weekly schedule.
47. The method of any one of embodiments 1 to 46, wherein the one-week schedule is repeated one or more times.
48. The method of any one of embodiments 1 to 47, wherein the cancer or tumor is a lymphoproliferative disorder.
49. The method of embodiment 48, wherein the lymphoproliferative disorder is B cell lymphoma or leukemia.
50. The method of embodiment 48, wherein the lymphoproliferative disorder is T cell lymphoma or leukemia.
51. The method of embodiment 48, wherein the lymphoproliferative disorder is a Hodgkin's lymphoma, a non-Hodgkin's lymphoma, or Burkitt's lymphoma.
52. The method of any one of embodiments 1 to 47, wherein the cancer or tumor is a solid tumor.
53. The method of any one of embodiments 1 to 52, wherein the cancer or tumor is a Herpesviridae associated cancer.
54. The method of any one of embodiments 1 to 52, wherein the cancer or tumor is associated with human cytomegalovirus (CMV), Epstein-Barr virus (EBV), or herpes simplex virus 1 or 2 (HSV-1 or -2).
55. The method of any one of embodiments 1 to 52, wherein the cancer or tumor is associated with Epstein-Barr virus (EBV).
56. The method of any one of embodiments 1 to 52, wherein the cancer or tumor is an Epstein- Barr virus positive cancer or tumor.
57. The method of any one of embodiments 1 to 52, wherein the cancer or tumor comprises a latent Epstein-Barr virus infection.
58. The method of any one of embodiments 1 to 52, wherein the cancer or tumor is breast cancer, non-small cell lung cancer, melanoma, head and neck, lymphoepithelioma-like carcinoma, bladder, gastric cancer, nasopharyngeal carcinoma, leiomyosarcoma, or colorectal cancer.
59. The method of any one of embodiments 1 to 52, wherein the cancer or tumor is nasopharyngeal carcinoma, leiomyosarcoma, lymphoepithelioma-like carcinoma, or gastric cancer.
60. The method of embodiment 59, wherein the cancer or tumor is nasopharyngeal carcinoma.
61. A method of treating an EBV-associated cancer or tumor in an individual, the method comprising administering to the individual afflicted with the cancer or tumor an effective amount of: a) nanatinostat; b) aciclovir, ganciclovir, valaciclovir, valganciclovir, or famciclovir; c) and a PD-1/PD-L1 axis inhibitor.
62. The method of embodiment 61, wherein the PD-1/PD-L1 axis inhibitor comprises nivolumab, pembrolizumab, cemiplimab, atezolizumab, avelumab, and durvalumab.
63. The method of embodiment 61, wherein the PD-1/PD-L1 axis inhibitor comprises nivolumab, pembrolizumab, or cemiplimab.
64. The method of embodiment 61, wherein the PD-1/PD-L1 axis inhibitor comprises pembrolizumab.
65. The method of any one of embodiments 61 to 64, wherein the EBV-associated cancer is nasopharyngeal carcinoma, leiomyosarcoma, lymphoepithelioma-like carcinoma, or gastric cancer.
66. The method of any one of embodiments 61 to 65, wherein the EBV-associated cancer is nasopharyngeal carcinoma.

### EXAMPLES

The following illustrative examples are representative of embodiments of compositions and methods described herein and are not meant to be limiting in any way.

### Example 1

### Phase 1 Study of Nanatinostat and Valganciclovir Tolerability

A traditional 3+3 dose escalation design will be used to determine the RP2D of nanatinostat and valganciclovir; approximately 9 to 18 patients with RM-NPC will be enrolled; cohorts of 3 to 6 patients with RM-NPC will be enrolled sequentially at escalating nanatinostat doses starting with 20 mg daily on Days 1 to 4 per week with valganciclovir 900 mg daily. Any patients in screening when the last patient is enrolled in a cohort may also be enrolled if eligible. Dose escalation of nanatinostat will continue (30 mg, 40 mg daily, days 1-4 per week) until the RP2D is identified, up to a maximum of 40 mg daily on Days 1 to 4 per week. Because plasma EBV DNA levels correlate closely with the presence of disease and response to therapy in RM-NPC, these data will be considered together with safety data in the selection of the RP2D.

Following dose escalation, up to 10 patients with advanced EBV+ non-NPC solid tumors (gastric cancer, lymphoepithelioma, and leiomyosarcoma) will be enrolled in an exploratory proof-of-concept cohort at the determined RP2D to further characterize the safety and PK of the combination in other solid tumors. Enrollment in the proof-of-concept cohort may be stopped before 10 patients are treated if a) evidence of safety or tolerability issues arise or b) once Phase 2 of the study completes enrollment.

### Example 2

### Phase 2 Study of Nanatinostat, Valganciclovir, and Pembrolizumab

Up to 60 patients with RM-NPC will be randomly assigned 1:1 to receive the RP2D of nanatinostat and valganciclovir with or without concomitant pembrolizumab to assess the preliminary anti-tumor activity, safety, and tolerability of each regimen. Randomization will be stratified by prior anti-PD-1 treatment exposure.

Phase 2 will have a sentinel cohort to assess the safety in each treatment arm prior to completing cohort enrollment. Patients will be randomly assigned 1:1 to both treatment groups (6 patients in each group; 12 patients total) during the sentinel cohort period. The starting dose of nanatinostat for both treatment groups will be the RP2D determined during Phase 1b on Days 1 to 4 of per week; all patients will be dosed with valganciclovir 900 mg daily. Pembrolizumab will be dosed at 200 mg IV every 3 weeks for those patients randomly assigned to the nanatinostat, valganciclovir, and pembrolizumab group.

The sentinel cohort period will be monitored by a Safety Monitoring Committee comprising the lead study Investigators and the Sponsor's Medical Monitor. When the first 6 patients in each treatment group of the safety sentinel cohort have completed 2 cycles (7 weeks) of treatment (or discontinued earlier), the Safety Monitoring Committee will review the clinical and laboratory data for any DLTs and/or emerging new or unexpected toxicities associated with the nanatinostat dose used in combination with valganciclovir and pembrolizumab to determine the next steps for dosing and continued enrollment. Recruitment will be paused until a safety data review is performed in the following situations:
1. If treatment was postponed due to the reasons listed in Section 5.5.4.2 for more than 2 of the first 6 enrolled patients who received at least one dose of any study drug.
2. If treatment was postponed due to the reasons listed in Section 5.5.4.2 for 4 or more of the first 9 patients who have received at least one dose of any study drug.

All patients will be monitored at weekly intervals for the first 6 weeks, and then at 3 week intervals thereafter starting at Week 8.

Tumor responses will be assessed at Week 8 and then every 6 weeks for the first 26 weeks (6 months) and every 12 weeks thereafter by the Investigator per Response Evaluation Criteria in Solid Tumors (RECIST) version 1.1 (v1.1).

Patients will continue to receive study treatment until the development of progressive disease (per Investigator assessment), unacceptable toxicity, withdrawal of consent, Investigator's discretion, initiation of new antineoplastic therapy, or study termination by the Sponsor. The maximum treatment with pembrolizumab is 24 months.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

What is claimed is:

1. A method of treating a cancer or tumor in an individual, the method comprising administering to the individual afflicted with the cancer or tumor an effective amount of:
a) a histone deacetylase inhibitor (HDACi); b) a nucleoside analog; c) and a checkpoint inhibitor antagonist.

2. The method of claim 1, wherein the HDACi comprises vorinostat, romidepsin, mocetinostat, belinostat, pracinostat, givinostat, panobinostat, CUDC-101, zabinostat, chidamide, domatinostat, entinostat, and combinations thereof.

3. The method of claim 1 wherein the HDACi comprises nanatinostat.

4. The method of claim 3, wherein the nanatinostat is administered at a dose of about 5 milligrams to about 160 milligrams per day.

5. The method of claim 3, wherein the nanatinostat is administered at a dose of about 5 milligrams to about 80 milligrams per day.

6. The method of claim 3, wherein the nanatinostat is administered at a dose of about 5 milligrams to about 40 milligrams per day.

7. The method of claim 3, wherein the nanatinostat is administered at a dose of about 5 milligrams to about 20 milligrams per day.

8. The method of claim 3, wherein the nanatinostat is administered at a dose of about 10 milligrams to about 20 milligrams per day.

9. The method of claim 3, wherein the nanatinostat is administered at a dose of about 10 milligrams to about 40 milligrams per day.

10. The method of claim 3, wherein the nanatinostat is administered at a dose of about 10 milligrams per day.

11. The method of claim 3, wherein the nanatinostat is administered at a dose of about 20 milligrams per day.

12. The method of claim 3, wherein the nanatinostat is administered at a dose of about 40 milligrams per day.

13. The method of claim 12, wherein the nanatinostat is administered orally.

14. The method of claim 13, wherein the nanatinostat is administered b.i.d.

15. The method of claim 13, wherein the nanatinostat is administered q.d.

16. The method of claim 1, wherein the nucleoside analog is a substrate of a viral thymidine kinase.

17. The method of claim 1, wherein the nucleoside analog comprises valganciclovir, ganciclovir, acyclovir, valaciclovir, famciclovir or a combination thereof.

18. The method of claim 1, wherein the nucleoside analog comprises valganciclovir.

19. The method of claim 18, wherein the valganciclovir is administered at dose of about 900 milligrams per day.

20. The method of claim 18, wherein the valganciclovir is administered at dose of about 450 milligrams per day.

21. The method of claim 1, wherein the checkpoint inhibitor antagonist is an antibody that binds to and inhibits the function of a checkpoint inhibitor.

22. The method of claim 1, wherein the checkpoint inhibitor comprises PD-1, PD-L1, PD-L2, or CTLA4.

23. The method of claim 22, wherein the checkpoint inhibitor comprises PD-1.

24. The method of claim 23, wherein the checkpoint inhibitor antagonist comprises nivolumab, pembrolizumab, cemiplimab, or a combination thereof.

25. The method of claim 22, wherein the checkpoint inhibitor comprises PD-L1.

26. The method of claim 25, wherein the checkpoint inhibitor antagonist comprises atezolizumab, avelumab, durvalumab, or a combination thereof.

27. The method of claim 22, wherein the checkpoint inhibitor comprises CTLA4.

28. The method of claim 27, wherein the checkpoint inhibitor antagonist comprises ipilimumab.

29. The method of claim 1, wherein the checkpoint inhibitor antagonist is administered intravenous.

30. The method of claim 1, wherein the checkpoint inhibitor antagonist is administered subcutaneous.

31. The method of claim 1, wherein the checkpoint inhibitor is administered at a dose of about 200 milligrams.

32. The method of claim 1, wherein the checkpoint inhibitor is administered at a dose of about 400 milligrams.

33. The method of claim 1, wherein the checkpoint inhibitor is administered at a dose of from about 1 milligram per kilogram to about 12 milligrams per kilogram.

34. The method of claim 1, wherein the checkpoint inhibitor is administered at a dose of about 2 milligrams per kilogram.

35. The method of claim 1, wherein the checkpoint inhibitor is administered at a dose of about 10 milligrams per kilogram.

36. The method of claim 1, wherein the checkpoint inhibitor is administered at a dose of about 300 milligrams.

37. The method of claim 1, wherein the checkpoint inhibitor is administered at a dose of about 240 milligrams.

38. The method of claim 1, wherein the checkpoint inhibitor is administered at a dose of about 350 milligrams.

39. The method of claim 1, wherein the checkpoint inhibitor is administered at a dose of about 480 milligrams.

40. The method of claim 1, wherein the checkpoint inhibitor is administered once every two weeks.

41. The method of claim 1, wherein the checkpoint inhibitor is administered once every three weeks.

42. The method of claim 1, wherein the checkpoint inhibitor is administered once every four weeks.

43. The method of claim 1, wherein the checkpoint inhibitor is administered once every six weeks.

44. The method of claim 3, wherein the nanatinostat is administered on a weekly schedule and the individual is administered nanatinostat for 2 days of the weekly schedule.

45. The method of claim 3, wherein the nanatinostat is administered on a weekly schedule and the individual is administered the nanatinostat for 3 days of the weekly schedule.

46. The method of claim 3, wherein the nanatinostat is administered on a weekly schedule and the individual is administered the nanatinostat for 4 days of the weekly schedule.

47. The method of claim 44, wherein the weekly schedule is repeated one or more times.

48. The method of claim 1, wherein the cancer or tumor is a lymphoproliferative disorder.

49. The method of claim 48, wherein the lymphoproliferative disorder is B cell lymphoma or leukemia.

50. The method of claim 48, wherein the lymphoproliferative disorder is T cell lymphoma or leukemia.

51. The method of claim 48, wherein the lymphoproliferative disorder is a Hodgkin's lymphoma, a non-Hodgkin's lymphoma, or Burkitt's lymphoma.

52. The method of claim 1, wherein the cancer or tumor is a solid tumor.

53. The method of claim 1, wherein the cancer or tumor is a Herpesviridae associated cancer.

54. The method of claim 1, wherein the cancer or tumor is associated with human cytomegalovirus (CMV), Epstein-Barr virus (EBV), or herpes simplex virus 1 or 2 (HSV-1 or -2).

55. The method of claim 1, wherein the cancer or tumor is associated with Epstein-Barr virus (EBV).

56. The method of claim 1, wherein the cancer or tumor is an Epstein-Barr virus positive cancer or tumor.

57. The method of claim 1, wherein the cancer or tumor comprises a latent Epstein-Barr virus infection.

58. The method of claim 1, wherein the cancer or tumor is breast cancer, non-small cell lung cancer, melanoma, head and neck, lymphoepithelioma-like carcinoma, bladder, gastric cancer, nasopharyngeal carcinoma, leiomyosarcoma, or colorectal cancer.

59. The method of claim 1, wherein the cancer or tumor is nasopharyngeal carcinoma, leiomyosarcoma, lymphoepithelioma-like carcinoma, or gastric cancer.

60. The method of claim 59, wherein the cancer or tumor is nasopharyngeal carcinoma.

61. A method of treating an EBV-associated cancer or tumor in an individual, the method comprising administering to the individual afflicted with the cancer or tumor an effective amount of: a) nanatinostat; b) aciclovir, ganciclovir, valaciclovir, valganciclovir, or famciclovir; c) and a PD-1/PD-L1 axis inhibitor.

62. The method of claim 61, wherein the PD-1/PD-L1 axis inhibitor comprises nivolumab, pembrolizumab, cemiplimab, atezolizumab, avelumab, and durvalumab.

63. The method of claim 61, wherein the PD-1/PD-L1 axis inhibitor comprises nivolumab, pembrolizumab, or cemiplimab.

64. The method of claim 61, wherein the PD-1/PD-L1 axis inhibitor comprises pembrolizumab.

65. The method of claim 61, wherein the EBV-associated cancer is nasopharyngeal carcinoma, leiomyosarcoma, lymphoepithelioma-like carcinoma, or gastric cancer.

66. The method of claim 61, wherein the EBV-associated cancer is nasopharyngeal carcinoma.
